(19) United States
(12) Patent Application Publication
(10) Pub. No.: US 2023/0377745 A1
(43) Pub. Date: Nov. 23, 2023

(54) METHOD OF ESTABLISHING A CLINICAL DECISION SUPPORT SYSTEM FOR SPC RISK EVALUATION AMONG PATIENTS WITH COLORECTAL CANCER USING A PREDICTION MODEL AND VISUALIZATION

(71) Applicants: Chi-Chang Chang, Taichung City (TW); Chi-Jie Lu, Taichung City (TW); Yi-Ju Tseng, Taichung City (TW); Ssu-Han Chen, Taichung City (TW); Chin-Jen Tseng, Taichung City (TW)

(72) Inventors: Chi-Chang Chang, Taichung City (TW); Chi-Jie Lu, Taichung City (TW); Yi-Ju Tseng, Taichung City (TW); Ssu-Han Chen, Taichung City (TW); Chin-Jen Tseng, Taichung City (TW)

(73) Assignee: Chung Shan Medical University, Taichung City (TW)

(21) Appl. No.: 17/746,268

(22) Filed: May 17, 2022

(51) Int. Cl. G16H 50/20; G16H 50/30

(52) U.S. Cl. CPC G16H 50/20; G16H 50/30

(57) ABSTRACT

A method of establishing a clinical decision support system for SPC risk evaluation among patients with colorectal cancer includes combining cancer characteristics into a characteristic assembly of SPC risk evaluation; obtaining clinical data of first participants to establish a database of SPC risk evaluation; entering the database into a machine learning algorithm; using the machine learning algorithms to establish a SPC risk evaluation model; using a characteristic interpreter to analyze the model; calculating a risk value of each cancer characteristic; presenting the risk values in graphics to establish a clinical decision support system; obtaining clinical data of second participants and inputting same into the clinical decision support system; using the machine learning algorithm for comparison and analysis; predicting risk for SPC; calculating a risk value of each cancer characteristic; presenting the risk values on the clinical decision support system; giving suggestions of decreasing risk; and monitoring changes of the risk.

### FIELD OF THE INVENTION

The invention relates to clinical decision support systems and more particularly to a method of establishing a clinical decision support system for SPC risk evaluation among patients with colorectal cancer using a prediction model and visualization so that a physician can evaluate risk of SPC among patients with colorectal cancer, make a correct clinical decision, and provide a patient appropriate advice.

### BACKGROUND OF THE INVENTION

A second primary cancer (SPC) is a second, unrelated cancer in a person who has previously experienced another cancer at any time. Both success rate of cancer treatment and survival rate increase due to effective cancer screening test and improved treatment. But the number of persons diagnosed with SPC also increases. SPC is the main cause of decreasing cancer survival rate. To the worse extent, SPC not only decreases the success rate of cancer treatment but also decreases quality of life of a patient with SPC. Thus, an early detection of SPC is critical to the disease-free survival in patients with cancer.

Currently, a patient can regularly take a cancer screening test with no item on SPC diagnosis. Therefore, risk of SPC of the patient cannot be evaluated. A patient may lose the chance of early finding of SPC. Furthermore, there is little clinical practice or technology on evaluating risk of SPC after colorectal cancer.

Therefore, it is necessary to provide a method of establishing a clinical decision support system for SPC risk evaluation among patients with colorectal cancer, in which a physician can use the method to evaluate risk of SPC after colorectal cancer, make a correct clinical decision, and give a patient appropriate advice.

### SUMMARY OF THE INVENTION

It is therefore one object of the invention to provide a method of establishing a clinical decision support system for SPC risk evaluation among patients with colorectal cancer using a prediction model and visualization comprising combining a plurality of cancer characteristics into a cancer characteristic assembly of SPC risk evaluation; obtaining clinical data of a plurality of first participants corresponding to the cancer characteristic assembly of SPC risk evaluation to establish a database of SPC risk evaluation; entering the database of SPC risk evaluation into a machine learning algorithm; using the machine learning algorithms to establish a SPC risk evaluation model; using a characteristics interpreter to analyze the SPC risk evaluation model; calculating a risk value of each cancer characteristics; presenting the risk values in graphics to establish a clinical decision support system with visualization; obtaining clinical data of a plurality of second participants corresponding to the characteristic assembly of SPC risk evaluation; inputting the clinical data into the clinical decision support system; using the machine learning algorithms for comparison and analysis; predicting risk for SPC; calculating a risk value of each cancer characteristics with respect to each patient; presenting the risk values on the clinical decision support system using visualization; giving suggestions of decreasing the risk for SPC with respect to each cancer characteristics; and monitoring changes of the risk for SPC based on the presentation shown on the clinical decision support system.

Preferably, the value of each cancer characteristic is a Shapley value or a significance of a feature.

The risk of SPC among patients with colorectal cancer is increased when the Shapley value is positive, and the risk of SPC among patients with colorectal cancer is decreased when the Shapley value is negative.

Preferably, the presentation is a bar chart, pie chart, line chart or any combination thereof.

The invention has the following advantages and benefits in comparison with the conventional art:

The method uses the cancer characteristic assembly of SPC risk evaluation and the machine learning algorithms to establish the SPC risk evaluation model, and finally establish the clinical decision support system using visualization so that a medical employee can do an overall evaluation of a patient. The medical employee can take into account many characteristics of the patient because the characteristic assembly of SPC risk evaluation includes different cancer characteristics, thereby greatly increasing correctness and effectiveness of SPC risk evaluation. By presenting the clinical decision support system using visualization, a clinical physician can conveniently and quickly make a clinical decision in a simple manner. The clinical decision support system can show value changes of each cancer characteristics with respect to the risk of SPC in real time. Therefore, the physician can evaluate the risk for SPC based on increased risk value and decreased risk value with respect to each cancer characteristics prior to giving a patient appropriate advice.

The above and other objects, features, and advantages of the invention will become apparent from the following detailed description taken with the accompanying drawings.

### BRIEF DESCRIPTION OF THE DRAWINGS

FIG. 1 is a flow chart of a method of the invention;

FIG. 2 schematically depicts interface simulation of a clinical decision support system according to a first preferred embodiment of the invention;

FIG. 3 shows risk values of cancer characteristics for the clinical decision support system of FIG. 2 in graphics;

FIG. 4 schematically depicts interface simulation of a clinical decision support system according to a second preferred embodiment of the invention; and

FIG. 5 shows risk values of cancer characteristics for the clinical decision support system of FIG. 4 in graphics.

### DETAILED DESCRIPTION OF THE INVENTION

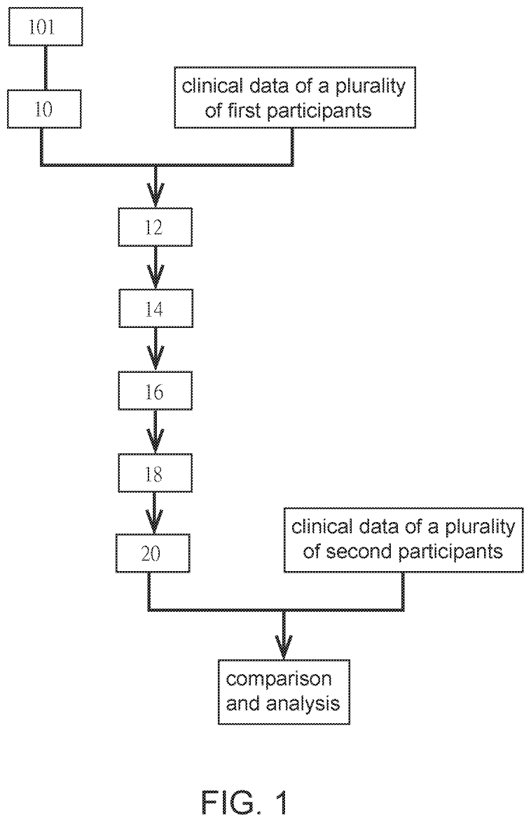

Referring to FIG. 1, a method of establishing a clinical decision support system for SPC risk evaluation among patients with colorectal cancer using a prediction model and visualization in accordance with the invention comprises the steps of combining a plurality of cancer characteristics 101 into a characteristic assembly 10 of SPC risk evaluation; obtaining clinical data of a plurality of first patients corresponding to the cancer characteristic assembly 10 of SPC risk evaluation to establish a database 12 of SPC risk evaluation; entering the database 12 of SPC risk evaluation into a machine learning algorithm 14; using the machine learning algorithm 14 to establish a SPC risk evaluation model 16; using a characteristic interpreter 18 to analyze the SPC risk evaluation model 16; calculating a risk value of each cancer characteristic 101; presenting the risk values in graphics to establish a clinical decision support system 20 with visualization; obtaining clinical data of a plurality of second participants corresponding to the characteristic assembly 10 of SPC risk evaluation; inputting the clinical data into the clinical decision support system 20; using the machine learning algorithm 14 for comparison and analysis; predicting risk for SPC; calculating a risk value of each cancer characteristic 101 with respect to each second participant; presenting the risk values on the clinical decision support system 20 using visualization; giving suggestions of decreasing the risk for SPC with respect to each cancer characteristic 101; and monitoring changes of the risk for SPC based on the presentation shown on the clinical decision support system 20.

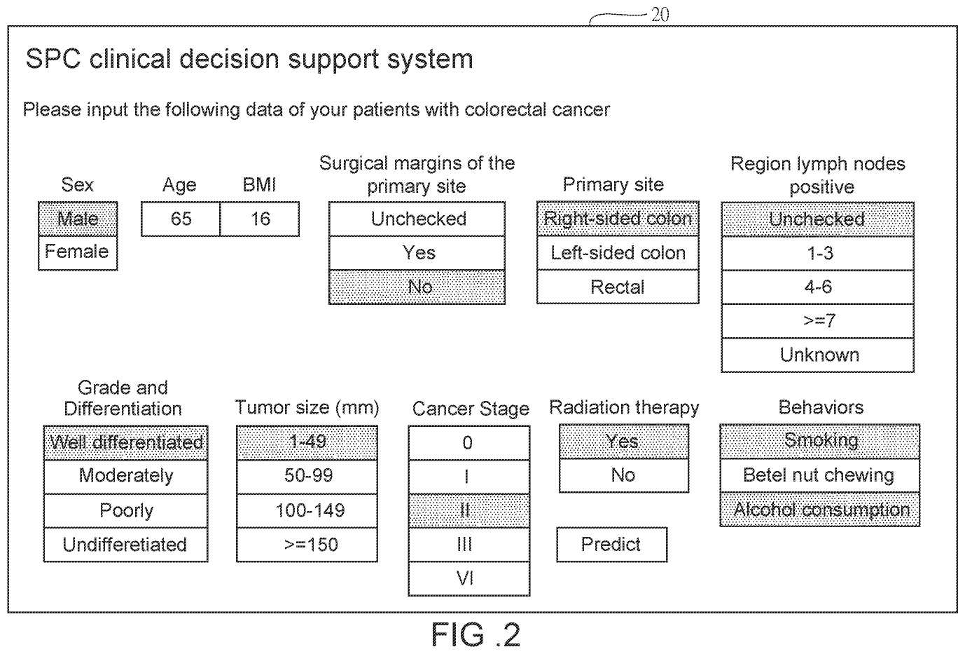

Referring to FIGS. 2 to 5 in conjunction with FIG. 1, the invention is discussed in detail based on clinical data of cancer characteristic assembly 10 of SPC risk evaluation with respect to colorectal cancer collected by a Taiwanese medical center.

Regarding admission and exclusion conditions of participants and number thereof, the participants are required to be colorectal cancer patients and no outside participants are recruited since the participants are required to be previous cancer patients of the medical center.

Retrospective period of the embodiment is from Jan. 1, 2004 to Dec. 31, 2018.

The method comprises:
Step 1 of collecting cancer data of 32,990 participants who have colorectal cancer in which the characteristic assembly 10 of SPC risk evaluation comprises 42 cancer characteristics 101 including diagnosis age, sex, primary site, tissue type, sexual orientation code, grade and differentiation, tumor size, positive region lymph nodes, clinical cancer stage T, clinical cancer stage N, clinical cancer stage M, clinical stage, pathology cancer stage T, pathology cancer stage N, pathology cancer stage M, pathology stage, combined states, surgery, surgical margins of the primary site, radiation therapy target range abstract, radiation therapy equipment, radiation therapy, surgery and radiation therapy order, the highest radiation dose, times of radiation therapy having the highest radiation dose, the lowest radiation dose, times of radiation therapy having the lowest radiation dose, overall therapy, body mass index (BMI), smoking behavior, betel nut chewing, alcohol consumption, SSF1 (carcinoembryonic antigen (CEA) lab value), SSF2 (carcinoembryonic antigen (CEA), the difference between lab value and normal value), SSF3 (tumor regression grade or score), SSF4 (circumferential resection margin, CRM), SSF5 (peritoneal invasion), SSF6 (KRAS mutation), SSF7 (obstruction), SSF8 (perforation), SSF9 (rectal tumor distance from anus), and cancer order;
Step 2 of obtaining clinical data of 32,990 first participants corresponding to the characteristic assembly 10 of SPC risk evaluation to establish a database 12 of SPC risk evaluation; entering the database 12 of SPC risk evaluation into a machine learning algorithm 14; using the machine learning algorithm 14 to establish a plurality of basic classifiers; selecting the basic classifiers having different points of view out of the plurality of basic classifiers; obtaining a classification result; and establishing a SPC risk evaluation model 16 based on the selected basic classifiers;
Step 3 of using the characteristic interpreter (Classic Shapley Value Estimation, Shapley Additive Explanation (SNAP)) 18 to analyze the SPC risk evaluation model 16; and calculating a risk value (e.g., Shapley value) of each cancer characteristic 101; presenting the risk values in graphics to establish a clinical decision support system 20 with visualization;
Step 4 of using both the characteristic interpreter 18 and the SPC risk evaluation model 16 to render the clinical decision support system 20 to be interactive so that a medical employee can predict risk for SPC and the risk value of each cancer characteristic 101 based on each cancer characteristic 101 of each second participant and the SPC risk evaluation model 16, and present original clinical data, the predicted risk for SPC and the risk value of each cancer characteristic 101 on the clinical decision support system 20 using visualization.

Beneficial effects of the method of the invention are detailed below. Taking advantage of the characteristic interpreter 18, a physician can advise a person advice of predicted risk of SPC. For example, a person may have an increased risk of SPC because the primary cancer treatment is surgery. Fortunately, the risk of SPC is decreased because the person appropriately controls his or her BMI and does not smoke. Therefore, a physician can adjust system parameters (e.g., BMI) to monitor changes in risk for SPC. The physician can advise the person to reasonably decrease weight if the physician finds that BMI control can decrease the risk of SPC.

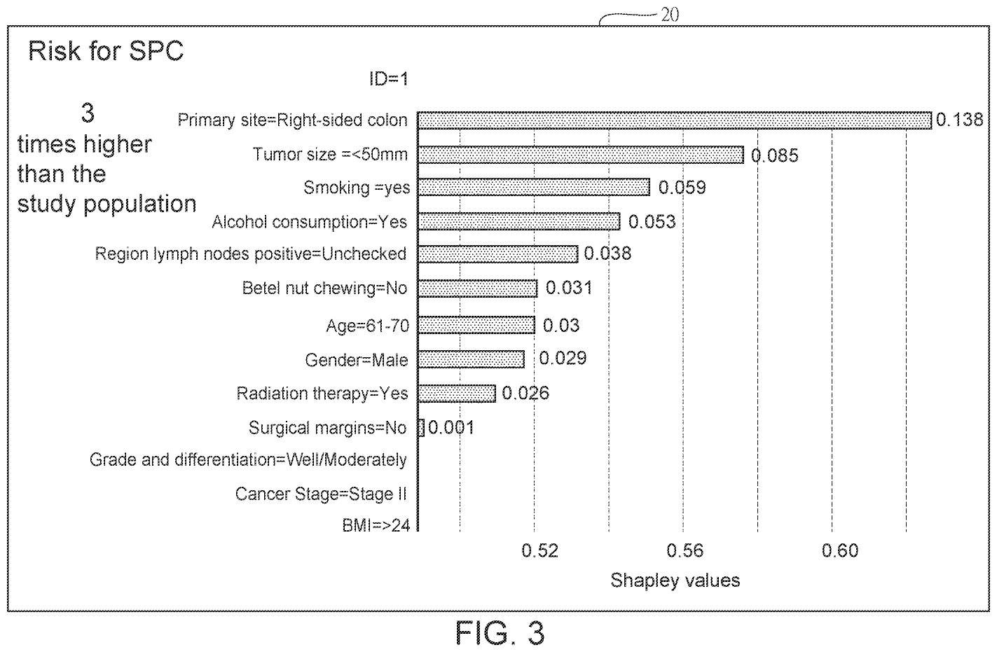

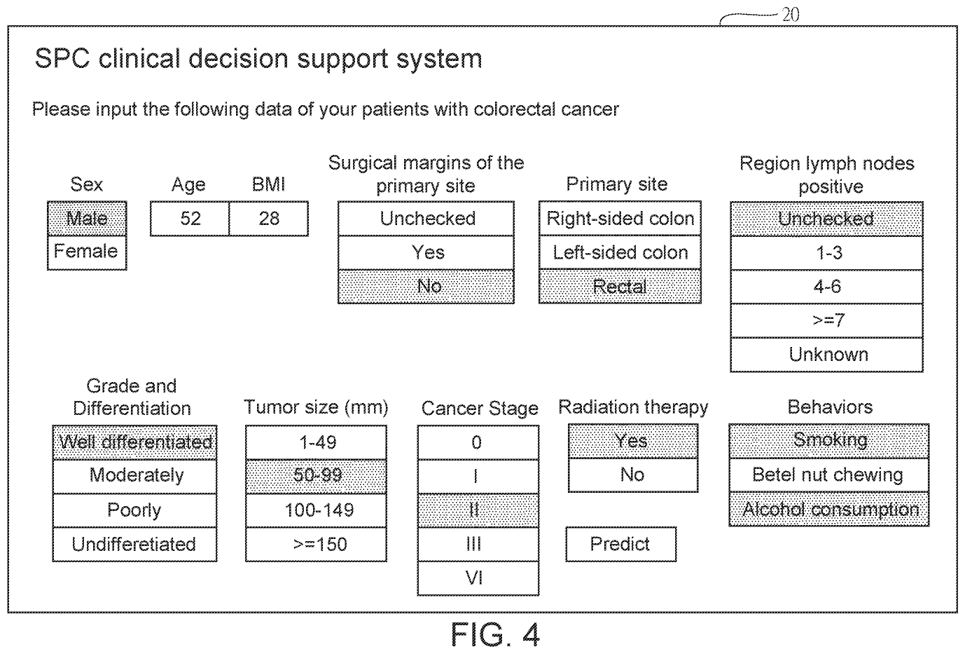

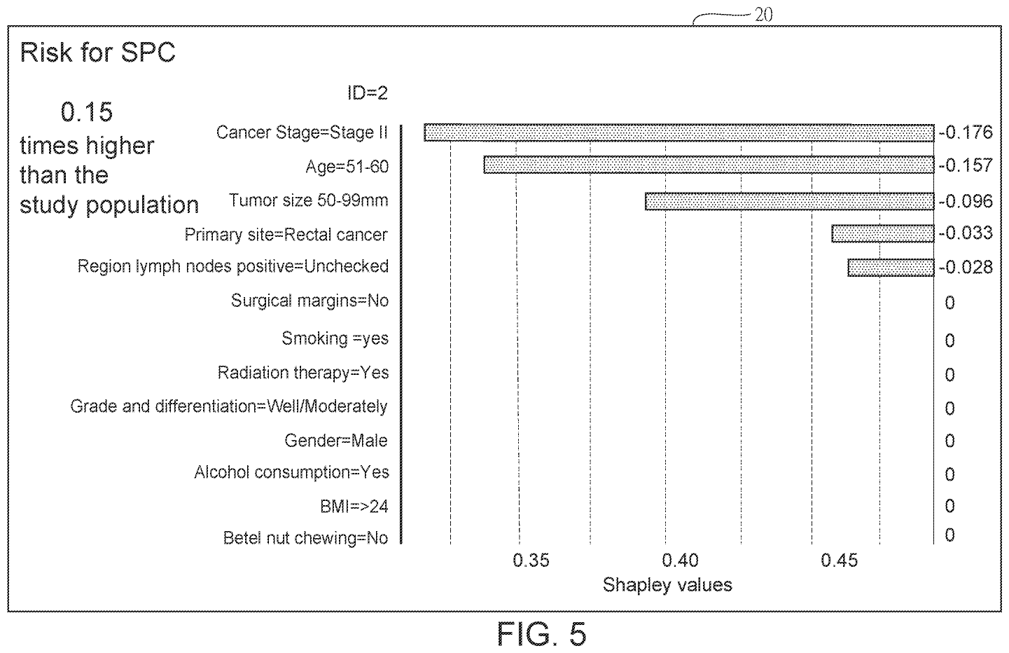

Interface simulations of the clinical decision support system 20 according to the first and second preferred embodiments with respect to different participants are shown in FIGS. 2 to 5 in which the interface simulation of the clinical decision support system 20 with respect to the first participant is shown in FIGS. 2 and 3, and the interface simulation of the clinical decision support system 20 with respect to the second participant is shown in FIGS. 4 and 5 respectively.

As shown in FIG. 2 specifically, a medical employee can input clinical data of the first participant into the clinical decision support system 20. Details of the data are shown below. Gender is male. Age is 65. BMI is 16. Surgical margins of the primary site are no. Primary site is the right colon. Positive lymph node region is unchecked. Grade and differentiation are well differentiated. Tumor size is 1-49 mm. Cancer stage is second. Radiation therapy is yes. Behaviors such as smoking and alcohol consumption are highlighted.

After the prediction button has been clicked, details of risk for SPC are shown in FIG. 3 in which the risk for SPC occurrence of the first participant with colorectal cancer is three times higher than the study population and is shown to the left, and a bar chart of Shapley values of the cancer characteristics 101 are shown to the right. Thus, a physician can quickly and correctly understand the risk of SPC of each cancer characteristic 101. Further, the risk of SPC among patients with colorectal cancer increases when the Shapley value is positive, and the risk of SPC among patients with colorectal cancer decreases when the Shapley value is negative.

Regarding each cancer characteristic 101 of the first participant, the primary site, which is the right colon, has a highest Shapley value of 0.138. It means that the primary site, the right-sided colon increases the risk of SPC of the first participant and has the greatest impact. Shapley values representing the risk for SPC of other cancer characteristics 101 (e.g., tumor size, smoking, alcohol consumption, betel nut chewing, age, gender, radiation therapy and surgical margins of the primary site) are gradually decreased. The physician can give advice to the first participant based on the risk values of the cancer characteristics 101 so as to help the first participant to decrease the risk of SPC. In addition, the physician can adjust parameters of one or more of the cancer characteristics 101 to monitor changes of the risk for SPC thereof.

As shown in FIG. 4 specifically, the medical employee can input clinical data of the second participant into the clinical decision support system 20. Details of the data are shown below. Gender is male. Age is 52. BMI is 28. Surgical margins of the primary site are no. Primary site is rectal. Positive lymph node region is unchecked. Grade and differentiation are well differentiated. Tumor size is 50-99 mm. Cancer stage is second. Radiation therapy is yes. Behaviors such as smoking and alcohol consumption are highlighted.

After the prediction button has been clicked, details of risk for SPC are shown in FIG. 5 in which the risk of SPC occurrence of the second participant with colorectal cancer is 0.15 times higher than the study population and is shown to the left, and the bar chart of Shapley values of the cancer characteristics 101 is shown to the right.

Regarding each cancer characteristic 101 of the second participant, the cancer stage has a lowest Shapley value of −0.176. This means that the second stage of the cancer decreases the risk for SPC of the second participant and has the greatest impact. Shapley values that represent the risk of SPC of other cancer characteristics 101 (e.g., age, tumor size, primary site, and region lymph nodes positive) are gradually increased.

It is envisaged by the invention that a medical employee can take many cancer characteristics of a patient into consideration prior to evaluating the risk for SPC and making a correct clinical decision.

Preferably, the machine learning algorithm 14 uses logistic regression, multivariate adaptive regression splines (MARS), decision tree classifiers, rule-based classifier, nearest neighbor classifiers, naïve Bayes classifier, artificial neural network, deep learning, support vector machine (SVM), random forest, eXtreme Gradient Boosting (XGBoost), categorical boosting, light gradient boosting machine (light GBM), ensemble learning methods, bagging and boosting-based classifiers, adaptive boosting-based classifiers, fuzzy set-based classifiers, genetic algorithms-based (GA-based) classifiers, genetic programming-based (GP-based) classifiers, meta heuristic-based classifiers, linear and nonlinear discriminant analysis, or any combination thereof.

Preferably, the cancer characteristic interpreter 18 includes local interpretable model-agnostic explanations (LIME), deep learning important features (DeepLIFT), layer-wise relevance propagation (LRP), Classic Shapley Value Estimation, Shapley Additive Explanation (SNAP), Shapley value-based model explanations, or any combination thereof.

Preferably, the cancer characteristic assembly 10 of SPC risk evaluation includes sex, birth year, initial data of first diagnosis, initial date of pathology diagnosis, method of confirming cancer, primary site, handedness, tissue type, sexual orientation code, grade and differentiation, clinical tumor size, pathology tumor size, number of checked region lymph nodes, positive region lymph nodes, distances of surgical margins and tumor cells, surgical margins of the primary site, cancer stage version, clinical cancer stage T, clinical cancer stage N, clinical cancer stage M, clinical cancer stage, pathology cancer stage T, pathology cancer stage N, pathology cancer stage M, pathology cancer stage, surgical therapy for tumor primary site by hospital, method of surgery for tumor primary site by hospital, surgical range of region lymph nodes by hospital, date of initial surgery, radiation therapy at the primary site by hospital, method of radiation therapy at the primary site, radiation dose for external body part radiation therapy at primary site, number of external body part radiation therapy, date of first external body part radiation therapy by hospital, date of final external body part radiation therapy by hospital, proximity radiation therapy by hospital, dose of proximity radiation therapy, chemotherapy performed by hospital, synchronous chemotherapy and radiation therapy, method of chemotherapy, times of performed chemotherapy, initial date of chemotherapy performed by hospital, hormone therapy by hospital, initial date of hormone therapy by hospital, date of final correspondence or death date, existence status, cancer status, date of first reoccurrence of SPC, type of first reoccurrence of SPC, causes of death, carcinoembryonic antigen (CEA) value, tumor decrease grade, pathology annular removal margins, nerve incursion, Kirsten rat sarcoma virus (KRAS) value, finding of intestine blockage or not before or after surgery, finding of intestine perforation or not before or after surgery, or any combination thereof.

Although the invention has been described in terms of preferred embodiments, those skilled in the art will recognize that the invention can be practiced with modifications within the spirit and scope of the appended claims.

What is claimed is:

1. A method of establishing a clinical decision support system for second primary cancer (SPC) risk evaluation among patients with colorectal cancer using a prediction model and visualization, comprising:
combining a plurality of cancer characteristics into a characteristic assembly of SPC risk evaluation;
obtaining clinical data of a plurality of first participants corresponding to the characteristic assembly of SPC risk evaluation to establish a database of SPC risk evaluation;
entering the database of SPC risk evaluation into a machine learning algorithm;
using the machine learning algorithm to establish a SPC risk evaluation model;
using a characteristic interpreter to analyze the SPC risk evaluation model;
calculating a risk value of each cancer characteristic;
presenting the risk values in graphics to establish a clinical decision support system with visualization;
obtaining clinical data of a plurality of second participants corresponding to the characteristic assembly of SPC risk evaluation;
inputting the clinical data into the clinical decision support system;
using the machine learning algorithm for comparison and analysis;
predicting risk for SPC;
calculating a risk value of each cancer characteristic with respect to each second participant;
presenting the risk values on the clinical decision support system using visualization;
giving suggestions of decreasing the risk for SPC with respect to each cancer characteristic; and
monitoring changes of the risk for SPC based on the presentation shown on the clinical decision support system.

2. The method of claim 1, wherein the machine learning algorithm uses logistic regression, multivariate adaptive regression splines (MARS), decision tree classifiers, rule-based classifier, nearest neighbor classifiers, naïve Bayes classifier, Bayesian networks, artificial neural network, deep learning, support vector machine (SVM), random forest, eXtreme Gradient Boosting (XGBoost), categorical boosting, light gradient boosting machine (light GBM), ensemble learning methods, bagging and boosting-based classifiers, adaptive boosting-based classifiers, fuzzy set-based classifiers, genetic algorithms-based (GA-based) classifiers, genetic programming-based (GP-based) classifiers, meta heuristic-based classifiers, linear and nonlinear discriminant analysis, or any combination thereof.

3. The method of claim 1, wherein the characteristic interpreter is local interpretable model-agnostic explanations (LIME), deep learning important features (DeepLIFT), layer-wise relevance propagation (LRP), Classic Shapley Value Estimation, Shapley Additive Explanation (SNAP), Shapley value-based model explanations, or any combination thereof.

4. The method of claim 1, wherein the characteristic assembly of SPC risk evaluation includes sex, birth year, initial data of first diagnosis, initial date of pathology diagnosis, method of confirming cancer, primary site, handedness, tissue type, sexual orientation code, grade and differentiation, clinical tumor size, pathology tumor size, number of checked region lymph nodes, positive region lymph nodes, surgical margins and tumor cells, surgical margins of the primary site, cancer stage version, clinical cancer stage T, clinical cancer stage N, clinical cancer stage M, clinical cancer stage, pathology cancer stage T, pathology cancer stage N, pathology cancer stage M, pathology cancer stage, surgical therapy for tumor primary site by hospital, method of surgery for tumor primary site by hospital, surgical range of region lymph nodes by hospital, date of initial surgery, radiation therapy at the primary site by hospital, method of radiation therapy at the primary site, radiation dose for external body part radiation therapy at primary site, number of external body part radiation therapy, date of first external body part radiation therapy by hospital, date of final external body part radiation therapy by hospital, proximity radiation therapy by hospital, dose of proximity radiation therapy, chemotherapy performed by hospital, synchronous chemotherapy and radiation therapy, method of chemotherapy, times of performed chemotherapy, initial date of chemotherapy performed by hospital, hormone therapy by hospital, initial date of hormone therapy by hospital, date of final correspondence or death date, existence status, cancer status, date of first reoccurrence of SPC, type of first reoccurrence of SPC, causes of death, carcinoembryonic antigen (CEA) value, tumor decrease grade, pathology annular removal margins, nerve incursion, Kirsten rat sarcoma virus (KRAS) value, finding of intestine blockage or not before or after surgery, finding of intestine perforation or not before or after surgery, or any combination thereof.

5. The method of claim 4, wherein the clinical cancer stage is clinical cancer stage T, clinical cancer stage N, or clinical cancer stage M.

6. The method of claim 4, wherein the pathological tumor is pathological cancer stage T, pathological cancer stage N, or pathological cancer stage M.

7. The method of claim 1, wherein the risk value of each cancer characteristic is a Shapley value or a significance of a feature.

8. The method of claim 7, wherein the risk of SPC among patients with colorectal cancer is increased when the Shapley value is positive, and the risk of SPC among patients with colorectal cancer is decreased when the Shapley value is negative.

9. The method of claim 1, wherein the presentation is a bar chart, pie chart, line chart or any combination thereof.
